(19) United States
(12) Patent Application Publication
(10) Pub. No.: US 2021/0402009 A1
(43) Pub. Date: Dec. 30, 2021

(54) CARBON NANOTUBE COMPOSITE VECTOR HAVING SYNERGISTIC EFFECT OF PHOTOTHERMAL THERAPY AND GENE THERAPY, PREPARATION METHOD THEREFOR AND APPLICATION THEREOF

(71) Applicant: DALIAN MINZU UNIVERSITY, Dalian (CN)

(72) Inventors: Yinan ZHAO, Dalian (CN); Shubiao ZHANG, Dalian (CN); Yingnan CAO, Dalian (CN); Quan ZHOU, Dalian (CN); Shaohui CUI, Dalian (CN); Defu ZHI, Dalian (CN); Huiying CHEN, Dalian (CN)

(21) Appl. No.: 17/291,658

(22) PCT Filed: Jul. 31, 2019
(86) PCT No.: PCT/CN2019/098652

§ 371 Date: May 6, 2021

(30) Foreign Application Priority Data
Nov. 6, 2018 (CN) 201811314938.5

(51) Int. Cl. A61K 48/00; A61K 41/00; A61P 35/00; A61K 31/7048; A61K 31/415; A61K 31/352; A61K 31/05; A61K 31/7024

(52) U.S. Cl. CPC A61K 48/0033; A61K 41/0052; A61P 35/00; A61K 31/7024; A61K 31/415; A61K 31/352; A61K 31/05; A61K 31/7048

(57) ABSTRACT

A carbon nanotube composite vector having a synergistic effect of photothermal therapy and gene therapy, a preparation method therefor, and an application thereof. The vector includes a vector moiety and a gene, and the vector moiety includes carbon nanotubes, a peptide lipid, and/or an additive. A modifier is immobilized on the carbon nanotubes by a self-assembly process to prepare the composite vector that can carry and transfer the gene. The composite vector overcomes the problems that pure carbon nanotubes have poor water solubility, low biocompatibility, and poor gene carrying and transfer efficiency; moreover, the composite vector has higher photothermal conversion performances and gene transfer efficiency, reduces cytotoxicity of carbon nanotubes, and alleviates the problem of localized accumulation of carbon nanotubes. The synergistic effect of photothermal therapy and gene therapy is applied to resolve the problem in tumor treatment that the efficacy of a single treatment method is poor.

### TECHNICAL FIELD

The present invention relates to the field of novel pharmaceutical preparations in the field of tumor treatment, and specifically to preparation and applications of carbon nanotube composite vectors capable of implementing combined photothermal and gene therapy.

### BACKGROUND

With the development of science and technology, some new methods and technologies for treating malignant tumors have emerged, among which photothermal therapy (PTT) technology and gene therapy technology have received extensive attention from researchers. PTT is a tumor treatment, in which a material having high photothermal conversion efficiency is injected into a human body and then gathers near the tumor tissue by using targeted recognition technology, and light energy is converted into heat energy under the irradiation of external near-infrared light to increase the temperature of a tumor site, so as to make use of the thermal killing effect caused by local overheating and secondary effects thereof. The mechanism is that the Bid protein in the cytoplasm is to be cleaved at a high temperature to form active molecules tBid, and the active molecules tBid enter the mitochondria, causing release of cytochrome C from the mitochondria, amplifying an apoptosis signal, activating Caspase9 and Caspase3 proteins, and thus inducing tumor cell apoptosis. This method avoids damage to normal tissue cells by performing near-infrared targeted irradiation on the tumor site to kill tumor cells, and is an effective new method for tumor treatment. However, in the treatment process, heat energy not only kills the tumor tissue, but also affects surrounding normal tissue cells; moreover, inflammation accompanying the treatment is also one of the defects of this treatment method.

The key to PTT is the performance of the photothermal material and whether the material can be concentrated in a lesion. With the continuous deepening of research, a variety of photothermal conversion materials have emerged. The carbon nanotube, as a reliable photothermal material, plays an important role in the field of PTT. The carbon nanotube is a tubular carbon molecule, having the diameter ranging from one nanometer to tens of nanometers, and the length ranging from a few nanometers to half a meter. The SP² hybridization of carbon atoms of the carbon nanotube and the arrangement of carbon-carbon σ bonds make the carbon nanotube widely applied in medicine, because it can adsorb a variety of drugs and easily penetrate cell membranes, thereby achieving the purpose of carrying and transferring drugs to enhance the efficacy of the drugs. However, in practical applications, there may be the problem of in vivo agglomeration and accumulation.

Gene therapy achieves the purpose of treatment by introducing an exogenous gene into cells of a patient to correct a defective gene. One of the keys to gene therapy is the construction of a gene transfer vectors. Non-viral gene vectors have attracted widespread attention due to the safety and low toxicity. A peptide lipid is a surfactant carrying positive charges on the head. Because the structure in which the head is hydrophilic and the tail is hydrophobic has been used to prepare cationic liposomes by self-assembly, drugs and genes can be effectively transferred. However, low transfer efficiency of peptide lipid vectors has always been an urgent problem to be solved.

It is difficult to achieve an excellent therapeutic effect if only gene therapy or photothermal therapy is used, and the current research trend is to implement multiple treatment methods at the same time.

### SUMMARY

In order to improve the gene transfer capability of existing transfection reagents and enhance the biocompatibility of carbon nanotubes, the present invention provides a novel carbon nanotube composite vector for cancer and tumor treatment.

The inventive concept of the present invention is: by non-covalent modification on carbon nanotubes by a modifier such as a peptide lipid, the carbon nanotubes can be loaded with a gene for gene therapy, where the specific performance is improved; moreover, photothermal therapy is combined by using the photothermal conversion efficiency of the carbon nanotubes to achieve the effect of combined gene and photothermal therapy.

The purpose of the present invention is achieved by the following technical solution: a carbon nanotube composite gene vector, composed of a vector moiety and a gene, where the vector moiety includes carbon nanotubes, a peptide lipid, and/or an additive. An N/P mass ratio of the vector moiety to the gene is 0.5:1 to 8:1. Preferably, the N/P ratio is 2:1 to 3:1. A molar ratio of the amount of the peptide lipid to the amount of the additive is 1:0.2 to 1:10. A mass ratio of the peptide lipid to the carbon nanotubes is 1:0.1 to 1:100, preferably, 1:0.5 to 1:5.

The additive is one or more of digoxin, celecoxib, quercetin, resveratrol, and a sucrose ester.

The gene is plasmid DNA, small interfering RNA, or Messenger RNA (mRNA).

The carbon nanotube is one or more of a multi-wall carbon nanotube, a single-wall carbon nanotube, a carboxylated multi-wall carbon nanotube, a carboxylated single-wall carbon nanotube, an aminated multi-wall carbon nanotube, an aminated single-wall carbon nanotube, a hydroxylated multi-wall carbon nanotube, and a hydroxylated single-wall carbon nanotube.

In the present invention, the efficiency of binding with the gene can be improved because the peptide lipid carries positive charges and a chemical bond on the surfaces of the carbon nanotubes can be easily bound to the gene; and digoxin, celecoxib, quercetin, and resveratrol can enhance the treatment of tumors by the vector. The present invention also provides a preparation method for a carbon nanotube composite vector, in which a peptide lipid, digoxin, celecoxib, quercetin, resveratrol, and a sucrose ester are fully adsorbed on the surface of the carbon nanotubes by using π bonds on the surfaces of the carbon nanotubes and a hydrophobic effect to form an aqueous dispersion of the carbon nanotubes, then a DNA or RNA diluent is slowly dripped into the dispersion of the carbon nanotubes, and the carbon nanotube composite vector is prepared by using an electrostatic effect.

The preparation method for the composite gene vector specifically includes: dissolving a peptide lipid and an additive, i.e., one or more of digoxin, celecoxib, quercetin, resveratrol, and a sucrose ester, into an organic solvent, uniformly dispersing the peptide lipid and additive on the surface of a container by a film dispersion process, performing vacuum drying for 12 to 36 h, preferably, for 24 h, slowly dripping an aqueous dispersion of carbon nanotubes and simultaneously performing ultrasonic oscillation at 50 to 60° C., then removing unbound and less bound carbon nanotubes by a high-speed centrifugation process, extracting a supernatant, mixing a vector moiety and a gene dilution at an N/P mass ratio of the vector moiety to a gene of 0.5:1 to 8:1, and preparing a preparation of the composite gene vector for photothermal therapy and gene therapy by an electrostatic compounding process.

The organic solvent is one or two of methanol and chloroform.

Another purpose of the present invention is to provide an application of a carbon nanotube composite gene vector in preparation of drugs or preparations for combined photothermal and gene tumor therapy.

The composite vector provided by the present invention can be injected into a human body, and then concentrated around a lesion by using passive targeting of the vector moiety; the temperature around the lesion is increased by irradiation of near-infrared light; tumor cells are killed by using thermal sensitivity of the tumor cells; the carried digoxin, celecoxib, quercetin, resveratrol, and sucrose ester will prevent the self-healing mechanism initiated by the tumor cells after heat damage; and photothermal treatment is combined. In vitro experiments of the carbon nanotube composite gene vector provided by the present invention show that it has capabilities of compressing and carrying DNA and RNA. After concentration around the tumor cells, the composite vector can pass through the cell membrane and enter the cytoplasm to release the carried DNA or RNA for gene therapy, thereby achieving the effect of combined photothermal and gene therapy. The construction of a vector system provides a new idea for tumor treatment, and has a great clinical application value. The composite vector overcomes the problems that pure carbon nanotubes have poor water solubility, low biocompatibility, and poor gene carrying and transfer efficiency; moreover, the composite vector has higher photothermal conversion performance and gene transfer efficiency, reduces cytotoxicity of carbon nanotubes, and alleviates the problem of localized accumulation of carbon nanotubes. The synergistic effect of photothermal therapy and gene therapy is applied to resolve the problem in tumor treatment that the efficacy of a single treatment method is poor.

### BRIEF DESCRIPTION OF THE DRAWINGS

FIG. 1 is a transmission electron microscope image of a carbon nanotube composite gene vector prepared.

FIG. 2 is a scanning electron microscope image of a carbon nanotube composite gene vector prepared.

FIG. 3 is an electrophoresis delay experiment diagram of a carbon nanotube composite gene vector and RNA carried thereby, in which a first lane is filled with markers (λ DNA-EcoR I+Hind III Markers), a second lane is naked RNA, and third to ninth lanes each are the composite vector/RNA having the compounding mass ratios of 1:1, 2:1, 4:1, 6:1, 8:1, and 16:1).

FIG. 4 is an experiment result diagram of carbon nanotube composite gene vector-induced hela apoptosis.

FIG. 5 is an experiment diagram of the effect of a carbon nanotube composite gene vector on the survival rate of hela cells.

FIG. 6 is an experiment result diagram of interference on A549 cell luciferase by a carbon nanotube composite gene vector.

FIG. 7 is a photothermal conversion diagram of a carbon nanotube composite gene vector when a 808 nm laser is used.

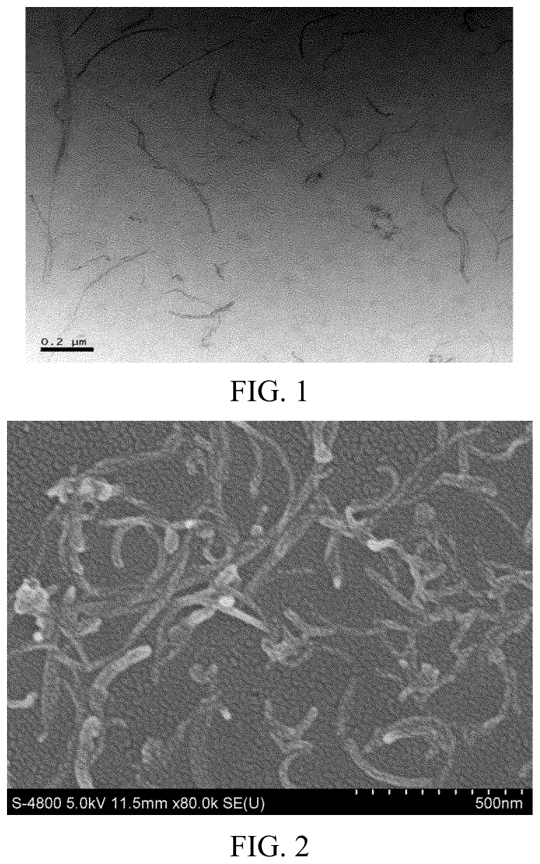

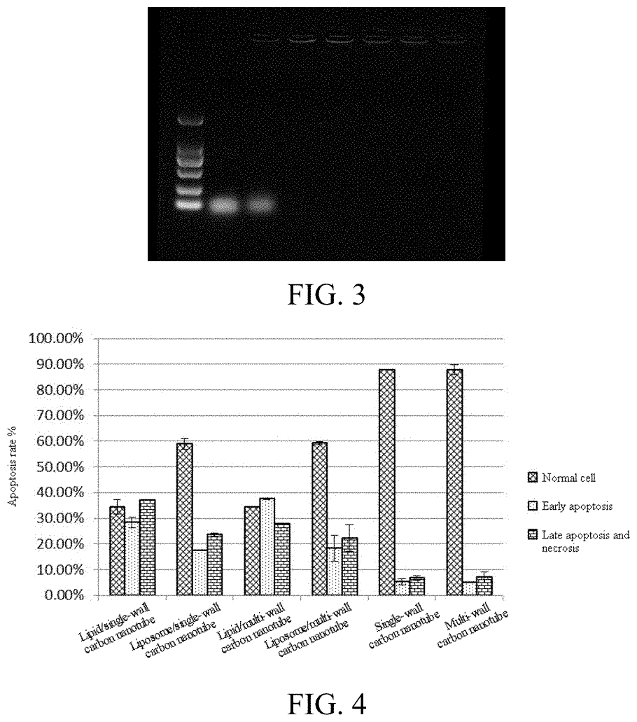

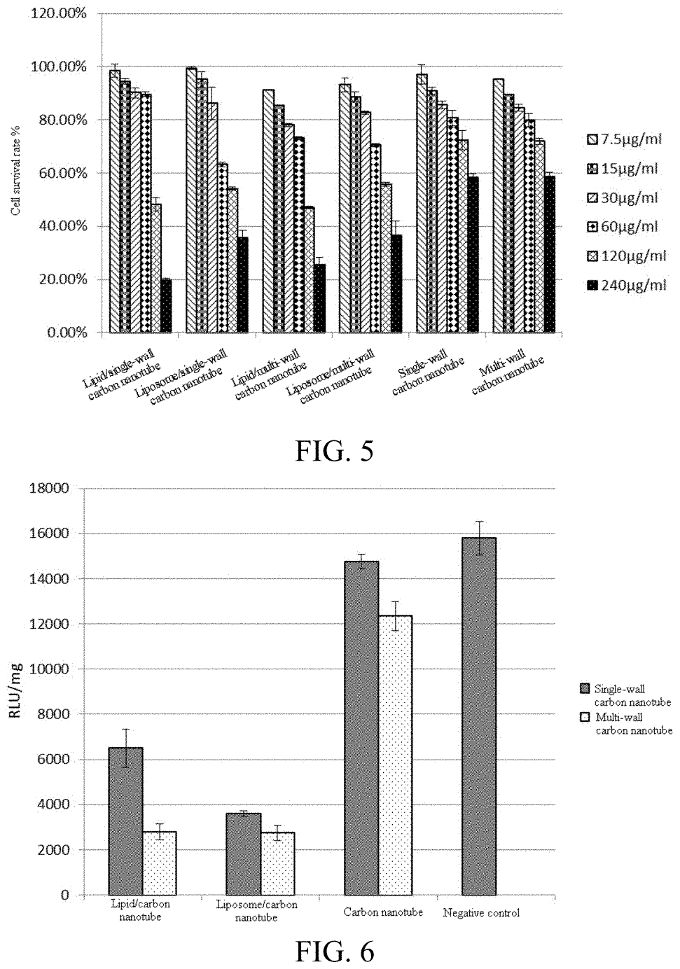

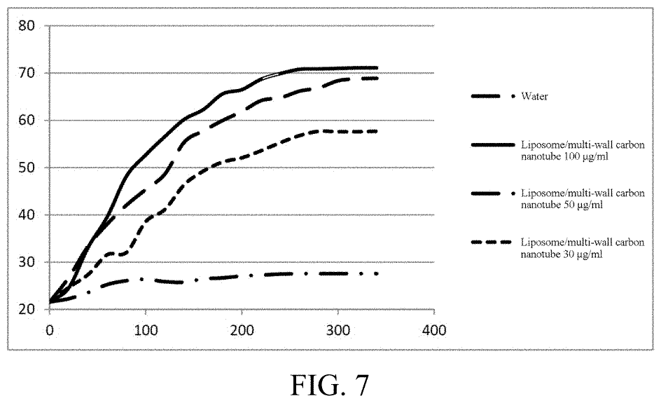

### DETAILED DESCRIPTION

The following drawings and specific examples are intended to illustrate the present invention in detail, but are not intended to limit the scope of protection of the present invention. Unless otherwise specified, the experimental methods used in the present invention are all conventional methods, and all used laboratory equipment, materials, reagents and the like can be purchased from chemical companies.

### Example 1

1 mg peptide lipid, 1 mg digoxin, and 1 mg resveratrol are weighed and dissolved in chloroform, the three reagents are uniformly dispersed on the surface of a container by using a nitrogen evaporator, and vacuum drying is performed for 24 h. 1 mg single-wall carbon nanotubes having diameters of 1 to 2 nm and lengths of 1 to 3 μm are weighed and ultrasonically dispersed in 1 ml ultrapure water, an aqueous dispersion of the carbon nanotubes is slowly dripped into the container coated with the three reagents, continuous ultrasonic oscillation is performed at 55° C. for 30 min, and a resulting suspension is centrifuged at 8000 r/min for 30 min to discard precipitate and extract a supernatant. The supernatant is slowly added into an RNA aqueous solution according to a metered N/P ratio of 3/1 and allowed to stand for 20 min, and a resulting composite vector is stored at 4° C.

### Example 2

1 mg peptide lipid, 0.4 mg quercetin, and 0.8 mg sucrose ester are weighed and dissolved in chloroform, the three reagents are uniformly dispersed on the surface of a container by using a nitrogen evaporator, and vacuum drying is performed for 36 h. 1 mg single-wall carbon nanotubes having diameters of 0.5 to 1 nm and lengths of 400 to 800 nm are weighed and ultrasonically dispersed in 1 ml ultrapure water, an aqueous dispersion of the carbon nanotubes is slowly dripped into the container coated with the three reagents, continuous ultrasonic oscillation is performed at 55° C. for 30 min, and a resulting suspension is centrifuged at 10000 r/min for 30 min to discard precipitate and extract a supernatant. The supernatant is slowly added into an RNA aqueous solution according to a metered N/P ratio of 2/1 and allowed to stand for 20 min, and a resulting composite vector is stored at 4° C.

### Example 3

1 mg peptide lipid is weighed and dissolved in 1 ml ultrapure water, 1.2 mg acidified multi-wall carbon nanotubes having diameters of 3 to 5 nm and lengths of 400 to 800 nm are weighed and ultrasonically dispersed in a peptide lipid solution, continuous ultrasonic oscillation is performed at 55° C. for 30 min, and a resulting suspension is centrifuged at 10000 r/min for 30 min to discard precipitate and extract a supernatant. The supernatant is slowly added into an RNA aqueous solution according to a metered N/P ratio of 4/1 and allowed to stand for 20 min, and a resulting composite vector is stored at 4° C.

### Example 4

1 mg peptide lipid and 0.2 mg sucrose ester are weighed and dissolved in chloroform, the two reagents are uniformly dispersed on the surface of a container by using a nitrogen evaporator, and vacuum drying is performed for 36 h. 1 mg single-wall carbon nanotubes having diameters of 0.5 to 1 nm and lengths of 400 to 800 nm are weighed and ultrasonically dispersed in 1 ml ultrapure water, an aqueous dispersion of the carbon nanotubes is slowly dripped into the container coated with the three reagents, continuous ultrasonic oscillation is performed at 55° C. for 30 min, and a resulting suspension is centrifuged at 10000 r/min for 30 min to discard precipitate and extract a supernatant. The supernatant is slowly added into an RNA aqueous solution according to a metered N/P ratio of 2/1 and allowed to stand for 20 min, and a resulting composite vector is stored at 4° C.

### Example 5

1 mg peptide lipid, 0.8 mg quercetin, and 1 mg sucrose ester are weighed and dissolved in chloroform, the three reagents are uniformly dispersed on the surface of a container by using a nitrogen evaporator, and vacuum drying is performed for 36 h. 1 mg multi-wall carbon nanotubes having diameters of 2 to 5 nm and lengths of 400 to 600 nm are weighed and ultrasonically dispersed in 1 ml ultrapure water, an aqueous dispersion of the carbon nanotubes is slowly dripped into the container coated with the three reagents, continuous ultrasonic oscillation is performed at 55° C. for 30 min, and a resulting suspension is centrifuged at 10000 r/min for 30 min to discard precipitate and extract a supernatant. The supernatant is slowly added into an RNA aqueous solution according to a metered N/P ratio of 4/1 and allowed to stand for 20 min, and a resulting composite vector is stored at 4° C.

### Example 6

In the present invention, cervical cancer cells Hela are selected as the research object; Hela cells are inoculated in a 12-well plate at a density of 1×10⁷/well and then cultured for 24 h in a DMEM culture solution having a 10% serum concentration; transfer is made to a serum-free low-glucose culture medium; a resulting composite vector is diluted in the serum-free low-glucose culture medium; transfection is enabled for 4 to 5 h; the culture medium having the composite vector is removed; the composite vector attached onto cell surfaces is cleaned using PBS; transfer is made to a DMEM culture solution having 10% serum and 4.5 g/l glucose; a laser having a 808 nm wavelength and 1 w/cm² power is used to vertically irradiate a cell culture plate to enable a photothermal conversion effect; the temperature of the cells is maintained between 40° C. and 43° C. for 5-10 min; cancer cell apoptosis is induced at a high temperature; and upon measurement 24 to 48 hours after the induction, the cell apoptosis efficiency is about 50% and less than 1% of the cells are killed at said use doses.

### Example 7

A vector moiety is compounded with FAM-siRNA having a fluorescent marker; Hela cells are introduced; the carbon nanotube composite gene vector of the present invention is detected in the cells using a flow cytometer 4 h after transfection; and the vector cellular uptake efficiency is higher than 80%.

### Example 8

In the present invention, lung adenocarcinoma cells A549 are selected as the research object; a vector moiety is compounded with Luc-siRNA that can interfere with expression of luciferase; the A549 cells are introduced; the expression level of luciferase in the cells is measured by using a microplate reader 4 h after transfection; and as shown in FIG. 6, experiments prove that the composite vector has a certain interference capability.

### Example 9

The mitochondria of living cells contain succinic acid dehydrogenasea, which are not contained in dead cells, and MTT can be reduced by the succinic acid dehydrogenasea in the living cells to produce water-insoluble blue-purple formazan. This property is applied in the present invention, i.e., MTT is used to stain Hela cells separately. Measurement is made by using a microplate reader. The number of the living cells relative to the dead cells can be indirectly reflected. It is found that the cell survival rate is higher under normal use metering, indicating that the composite vector of the present invention has lower toxicity.

### Example 10

The composite vector of the present invention is intravenously injected into a tumor-bearing mouse at a dose of 10 mg/kg once every two days; a laser device having a 808 nm wavelength and 3 w/cm² power is used for 5 min irradiation once every 24 h for a total of 7 days; the temperature of a lesion site is increased by the irradiation; the morphology of the lesion site is observed by using thermal infrared imaging; at the same time, small interfering RNA in the composite vector is used for gene therapy to achieve the purpose of treatment and auxiliary imaging. After daily irradiation, the tumor volume and the body weight of the mouse are measured. After the treatment is completed, the mouse is killed by cervical dislocation, and the tumor and other organs are taken out for pathological research.

The descriptions above are only preferable specific implementations of the present invention and creation. However, the scope of protection of the present invention and creation is not limited thereto. For a person skilled in the art, within the technical scope disclosed by the present invention and creation, any equivalent substitution or variation should be within the scope of protection of the present invention and creation according to the technical solution of the present invention and creation and the inventive concept thereof.

1. A carbon nanotube composite gene vector, composed of a vector moiety and a gene, wherein the vector moiety comprises carbon nanotubes, a peptide lipid, and/or an additive; an N/P mass ratio of the vector moiety to the gene is 0.5:1 to 8:1; a molar ratio of an amount of the peptide lipid to an amount of the additive is 1:0.2 to 1:10; and a mass ratio of the peptide lipid to the carbon nanotubes is 1:0.1 to 1:100.

2. The carbon nanotube composite gene vector according to claim 1, wherein the N/P mass ratio of the vector moiety to the gene is 2:1 to 3:1.

3. The carbon nanotube composite gene vector according to claim 1, wherein the mass ratio of the peptide lipid to the carbon nanotubes is 1:0.5 to 1:5.

4. The carbon nanotube composite gene vector according to claim 1, wherein the additive is one or more of digoxin, celecoxib, quercetin, resveratrol, and a sucrose ester.

5. The carbon nanotube composite gene vector according to claim 1, wherein the gene is a plasmid DNA, a small interfering RNA, or a Messenger RNA (mRNA).

6. The carbon nanotube composite gene vector according to claim 1, wherein the carbon nanotube is one or more of a multi-wall carbon nanotube, a single-wall carbon nanotube, a carboxylated multi-wall carbon nanotube, a carboxylated single-wall carbon nanotube, an aminated multi-wall carbon nanotube, an aminated single-wall carbon nanotube, a hydroxylated multi-wall carbon nanotube, and a hydroxylated single-wall carbon nanotube.

7. A preparation method for the carbon nanotube composite gene vector according to claim 1, comprising: dissolving a peptide lipid and an additive, i.e., one or more of digoxin, celecoxib, quercetin, resveratrol, and a sucrose ester, into an organic solvent, uniformly dispersing the peptide lipid and the additive on a surface of a container by a film dispersion process, performing vacuum drying for 12 to 36 h, slowly dripping an aqueous dispersion of carbon nanotubes, and simultaneously performing ultrasonic oscillation at 50 to 60° C., then removing unbound and less bound carbon nanotubes by a high-speed centrifugation process, extracting a supernatant, mixing a vector moiety and a gene dilution at an N/P mass ratio of the vector moiety to a gene of 0.5:1 to 8:1, and preparing the composite gene vector by an electrostatic compounding process.

8. The preparation method according to claim 7, wherein the organic solvent is one or two of methanol and chloroform.

9. An application of the carbon nanotube composite gene vector according to claim 1 in preparation of drugs or preparations for tumor photothermal and gene combined therapy.
